(19) United States
(12) Patent Application Publication
(10) Pub. No.: US 2011/0237840 A1
(43) Pub. Date: Sep. 29, 2011

(54) PROCESS FOR PREPARING POTASSIUM AND POTASSIUM COMPOUNDS

(75) Inventors: Hagen Wilmer, Ludwigshafen (DE); Andreas Keller, Speyer (DE)

(73) Assignee: BASF SE, Ludwigshafen (DE)

(21) Appl. No.: 13/071,989

(22) Filed: Mar. 25, 2011

Related U.S. Application Data
(60) Provisional application No. 61/317,712, filed on Mar. 26, 2010.

(51) Int. Cl. C07C 29/70; C22B 26/10

(52) U.S. Cl. 568/851; 75/589; 75/745

(57) ABSTRACT

The invention relates to a process for preparing potassium and potassium compounds

### DESCRIPTION

The invention relates to a process for preparing potassium and potassium compounds.

Potassium is obtained on an industrial scale by reaction of potassium chloride with sodium. However, the equilibrium of this endothermic reaction (ΔHᵣ=+25 kJ/mol) lies on the side of the starting materials and the boiling points of potassium and sodium of 759 and 883° C. are not very far apart, so that simple distillation is not sufficient to obtain sodium-free potassium. These problems are solved by a reactive rectification at about 800° C., as described in U.S. Pat. No. 2,480,655. The complex column structure and the associated capital and subsequent costs for carrying out the reactive rectification adversely effect the economics of the process.

DE 1932129 describes the preparation of potassium by reaction of potassium sulfate with iron in the presence of calcium oxide at temperatures of from 950 to 1300° C. The potassium formed is removed from the reaction mixture by distillation. The use of potassium hydroxide as starting material is described as disadvantageous because of the hygroscopic nature of potassium hydroxide; in addition, the high energy costs in this process are unfavorable.

The reaction of potassium fluoride with magnesium at temperatures of from 460 to 500° C. is likewise known (J.-P. Bastide et al., Eur. J. Solid State Inorg. Chem. 1989, vol. 26, pages 575 to 584). In this process too, the high energy costs are disadvantageous.

A further possible way of preparing metallic potassium is described in DE 3614698. Here, magnesium and potassium hydroxide react in an inert solvent to form potassium and magnesium oxide, with hydrogen being given off. The potassium can be isolated by further work-up steps: removal of the solvent, introduction of a liquid having a different density, decantation, filtration. Compared to the process of U.S. Pat. No. 2,480,655, this process has the advantage that it is a significantly exothermic reaction (ΔHᵣ=−176 kJ/mol), as a result of which the equilibrium lies on the product side. Disadvantages of this process are the additional steps for separating the potassium from the K/MgO/solvent mixture and the work-up of the auxiliaries obtained.

It was an object of the invention to provide a process for preparing potassium and potassium compounds which avoids the disadvantages of the known processes. The process should be able to be carried out at lower temperatures than the known processes in order to improve the economics of potassium production.

The object is achieved according to the invention by a process for preparing potassium by reacting potassium hydroxide with magnesium without solvent.

The invention therefore provides a process for preparing potassium by reacting potassium hydroxide with magnesium without solvent at a temperature in the range from 280 to 450° C.

In a preferred embodiment of the invention, the reaction of potassium hydroxide with magnesium takes place without solvent at a temperature in the range from 350 to 400° C.

In the process of the invention, the reactants potassium hydroxide and magnesium are used in a stoichiometric ratio of 1:1, corresponding to the reaction equation below.

KOH+Mg→K+MgO+0.5 H₂

In a preferred embodiment of the invention, an excess of magnesium is used in order to eliminate any water present as a result of the hygroscopic nature of potassium hydroxide. For example, the ratio of KOH to Mg is about 1:1.1.

A suitable reaction vessel for carrying out the process of the invention has to allow controlled release of the hydrogen formed. In addition, the reaction has to occur in an inert atmosphere in order to avoid subsequent reactions of potassium. For example, the reaction can be carried out under nitrogen or argon. A suitable reaction vessel is, for example, a stirred vessel or paddle dryer.

In a preferred embodiment of the invention, potassium hydroxide is placed in a reaction vessel and heated to the melting point of 360° C. Magnesium is subsequently added in portions in order to remove the hydrogen formed and the liberated heat in a controlled manner. As soon as the first potassium has been formed, the temperature can be reduced, for example to 280° C., since mixtures of potassium and potassium hydroxide are also liquid at temperatures below the melting point of potassium hydroxide.

The invention further provides a process for preparing potassium by reacting potassium hydroxide with magnesium without solvent in the presence of potassium at a temperature in the range from 63 to 450° C.

In a preferred embodiment of the invention, potassium and magnesium are placed in a reaction vessel at a temperature in the range from 63 to 360° C. and potassium hydroxide is then added in portions in order to remove the hydrogen formed and the liberated heat in a controlled manner. The amount of potassium present at the beginning of the reaction can be small relative to the amount of unreacted potassium hydroxide, for example in the range from 0.1 to 40% by weight, preferably in the range from 5 to 25% by weight.

In a further preferred embodiment of the invention, potassium is separated off and isolated from the reaction mixture by distillation or rectification. The distillation or rectification of the potassium can be carried out continuously or batchwise; it is possible to use a column and a two-stage or multistage condensation can be carried out. The distillation or rectification of the potassium can be carried out at atmospheric pressure in a temperature range from about 760 to 1100° C. or preferably under reduced pressure, for example at 1 mbar in a temperature range from about 330 to 700° C.

Owing to the large difference between the boiling points of the reactants (K: 759° C., KOH: 1320° C., Mg: 1107° C., MgO: ˜3600° C.) potassium having a very high purity, for example with an Mg content of less than 100 ppm, can be obtained by means of distillation or rectification.

The invention further provides a process for preparing potassium compounds, which comprises the steps
a) reaction of potassium hydroxide with magnesium without solvent at a temperature in the range from 280 to 450° C. or reaction of potassium hydroxide with magnesium without solvent in the presence of potassium at a temperature in the range from 63 to 450° C. and
b) reaction of the reaction mixture from step a) with an alcohol, a primary or secondary amine or ammonia in the presence of a solvent.

In a preferred embodiment of the invention, the reaction mixture from step a) is cooled, for example to room temperature, admixed with a solvent and subsequently reacted with an alcohol, a primary or secondary amine or ammonia.

Suitable solvents are stable toward metallic potassium, for example hydrocarbons or ethers. A preferred solvent is tetrahydrofuran (THF).

The alcohols and amines to be used according to the invention have the general formulae R¹OH and R¹R²NH, respectively, where R¹ can be C₁-C₂₄-alkyl, C₃-C₁₀-cyclo-alkyl, C₆-C₁₄-aryl, C₇-C₂₄-alkaryl or C₇-C₂₄-aralkyl and R² can be hydrogen, C₁-C₂₄-alkyl, C₃-C₁₀-cycloalkyl, C₆-C₁₄-aryl, C₇-C₂₄-alkaryl or C₇-C₂₄-aralkyl.

Examples of alkyl groups are methyl, ethyl, propyl, isopropyl, butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- or 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and isopinocampheyl. Preferred alkyl groups are methyl, ethyl, propyl, isopropyl, butyl, isobutyl, sec-butyl, tert-butyl, amyl, isoamyl, sec-amyl, 1,2-dimethylpropyl and 1,1-dimethylpropyl.

Examples of cycloalkyl groups are cyclopropyl, cyclobutyl, cyclopentyl, cyclohexyl, cycloheptyl, cyclooctyl, cyclononyl and cyclodecyl. Preferred cycloalkyl groups are cyclopropyl, cyclopentyl and cyclohexyl.

Examples of aryl groups are phenyl and naphthyl.

Examples of aralkyl groups are benzyl, 1- or 2-phenylethyl, 1-, 2- or 3-phenylpropyl, mesityl and 2-, 3- or 4-methylbenzyl.

Examples of alkaryl groups are 2-, 3- or 4-methylphenyl, 2-, 3- or 4-ethylphenyl and 2-, 3-, 4-, 5-, 6-, 7- or 8-methyl-1-naphthyl.

The process of the invention can be carried out at temperatures in the range from −50 to +65° C., preferably at room temperature. The hydrogen formed has to be discharged.

The potassium compounds formed generally dissolve in the solvent used and can be separated from undissolved magnesium oxide by suitable separation methods, for example by filtration.

### EXAMPLES

### Example 1

### Preparation of Potassium Without Solvent

589 kg of 95% pure KOH were placed in a nickel-plated stirred vessel having a volume of 3 m³ and heated to 370° C. The vessel was subsequently made inert by means of nitrogen and the stirrer was switched on. The speed of rotation was 80 revolutions per minute. 243 kg of magnesium were then introduced at a constant rate via a rotary valve over a period of 4 hours. During the first hour, the temperature of the reaction mixture was kept constant and the temperature was subsequently reduced quickly to 300° C. After the addition of magnesium was complete, the vessel was stirred for a further 30 minutes.

### Example 2

### Preparation of Potassium Without Solvent in the Presence of Potassium

A nickel-plated stirred vessel having a volume of 3 m³ was made inert by means of nitrogen and heated to 100° C. 100 kg of potassium were introduced into the stirred vessel. 243 kg of magnesium were subsequently introduced into the vessel via a rotary valve and the stirrer was switched on. The speed of rotation was 80 revolutions per minute. The mixture was heated to 250° C. over a period of 4 hours. 589 kg of 95% pure KOH were then added at a constant rate via a rotary valve over a period of 4 hours. After the addition of KOH was complete, the vessel was stirred for a further 30 minutes.

### Example 3

### Distillation of Potassium

The product mixture from Example 1 was drained into a paddle dryer which had been made inert by means of nitrogen and had been heated to 100° C. After the paddles had been started up, a vacuum of 10 mbar was applied and the mixture was subsequently heated to 750° C. over a period of 6 hours. The potassium which vaporized was condensed and collected in a vessel which had been made inert. The magnesium content of the potassium was less than 0.1% by weight.

### Example 4

### Distillation of Potassium

The product mixture from Example 2 was drained into a paddle dryer which had been made inert by means of nitrogen and had been heated to 100° C. After the paddles had been started up, a vacuum of 10 mbar was applied and the mixture was subsequently heated to 750° C. over a period of 6 hours. The potassium which vaporized was fractionally condensed, with the division into the fractions 1 and 2 being selected in a ratio of 3:1. The magnesium content of the first fraction is less than 0.01% by weight. The second fraction is conveyed into the nickel-plated stirred vessel and used for the next reaction batch.

### Example 5

### Preparation of Potassium Tert-Butoxide (KTB)

The product mixture from Example 1 was drained into a stirred vessel which had been made inert by means of nitrogen and was heated to 70° C. 3300 kg of THF were then added over a period of 2 hours. The temperature was set so that THF refluxed (about 66° C.). 590 kg of isopropanol were then added at a constant rate over a period of 4 hours, resulting in liberation of hydrogen. After the reaction, the mixture was stirred under reflux for a further 30 minutes and the temperature was then decreased to 30° C. The mixture was then filtered in order to separate the KTB/THF mixture from MgO.

1-9. (canceled)

10. A process for preparing potassium by reacting potassium hydroxide with magnesium, wherein the reaction takes place without solvent at a temperature in the range from 280 to 450° C.

11. The process according to claim 10, wherein the reaction takes at a temperature in the range from 350 to 400° C.

12. A process for preparing potassium by reacting potassium hydroxide with magnesium, wherein the reaction takes place without solvent in the presence of potassium at a temperature in the range from 63 to 450° C.

13. The process according to claim 12, wherein potassium is present in a ratio to the amount of unreacted potassium hydroxide in the range from 0.1 to 40% by weight at the beginning of the reaction.

14. The process according to claim 10, wherein potassium is separated from the reaction mixture by distillation or rectification.

15. The process according to claim 12, wherein potassium is separated from the reaction mixture by distillation or rectification.

16. A process for preparing potassium compounds, which comprises
a1) reacting potassium hydroxide with magnesium without solvent at a temperature in the range from 280 to 450° C. or
a2) reacting potassium hydroxide with magnesium without solvent in the presence of potassium at a temperature in the range from 63 to 450° C. and
b) reacting the reaction mixture from step a1) or a2) with an alcohol, a primary or secondary amine or ammonia in the presence of a solvent.

17. The process according to claim 16, wherein the alcohol and amine is a compound of the formulae R¹ OH and R¹R²NH, respectively, wherein
R¹ is C₁-C₂₄-alkyl, C₃-C₁₀-cycloalkyl, C₆-C₁₄-aryl, C₇-C₂₄-alkaryl or C₇-C₂₄-aralkyl and
R² is hydrogen, C₁-C₂₄-alkyl, C₃-C₁₀-cycloalkyl, C₆-C₁₄-aryl, C₇-C₂₄-alkaryl or C₇-C₂₄-aralkyl.

18. The process according to claim 16, wherein tetrahydrofuran is used as solvent.

19. The process according to claim 17, wherein tetrahydrofuran is used as solvent.

20. The process according to claim 16, wherein the reaction in step b) is carried out at temperatures in the range from −50 to +65° C.

21. The process according to claim 19, wherein the reaction in step b) is carried out at temperatures in the range from −50 to +65° C.

22. The process according to claim 16, wherein the reaction comprises step a1).

23. The process according to claim 21, wherein the reaction comprises step a1).

24. The process according to claim 16, wherein the reaction comprises step a2).

25. The process according to claim 21, wherein the reaction comprises step a2).
